(19) United States
(12) Patent Application Publication
(10) Pub. No.: US 2024/0167989 A1
(43) Pub. Date: May 23, 2024

(54) SYSTEM AND METHOD FOR TESTING OF FLUID CATALYTIC CRACKING CATALYSTS

(71) Applicant: Saudi Arabian Oil Company, Dhahran (SA)

(72) Inventors: Omer Refa KOSEOGLU, Dhahran (SA); Anas Farkad ALKHANI, Dhahran (SA)

(21) Appl. No.: 18/057,827

(22) Filed: Nov. 22, 2022

(51) Int. Cl. G01N 31/10; B01J 8/18; B01J 8/22; B01J 8/26; C10G 11/18; G01N 33/28

(52) U.S. Cl. CPC G01N 31/10; B01J 8/1809; B01J 8/22; B01J 8/26; C10G 11/18; G01N 33/28; B01J 2208/00628; B01J 2208/00707; C10G 2300/701

(57) ABSTRACT

Systems and methods for assessing FCC catalysts are provided, in particular FCC catalysts obtained from an operational FCC unit. The operator may test the FCC catalysts under real time conditions, enabling reductions in the amount of time necessary to assess the viability of the FCC catalyst from the operational FCC unit under different conditions, for different feeds, and/or with additional/different catalyst additives, resulting vast reductions in the cost of this aspect of FCC operations.

### TECHNICAL FIELD

The present disclosure relates to systems and methods for testing of fluidized catalytic cracking catalysts.

### DESCRIPTION OF RELATED ART

In fluidized catalytic cracking (FCC) processes, petroleum derived hydrocarbons are catalytically cracked within the presence of a catalyst maintained in a fluidized state, in which the catalyst is regenerated on a continuous basis. The main product from such processes has generally been gasoline. Other products are also produced in smaller quantities via conventional FCC processes such as liquid petroleum gas and cracked gas oil. Coke deposited on the catalyst is burned off at high temperatures and in the presence of air prior to recycling the regenerated catalyst back to the reaction zone.

Fluid catalytic cracking is the largest refining process used for gasoline production and converts heavy feedstocks such as vacuum distillates, atmospheric residues, and deasphalted oil into lighter products, which are rich in olefins and aromatics. FCC catalyst systems typically include solid acids of fine-particles such as zeolites, aluminum silicate, treated clay (kaolin), bauxite, and silica-alumina.

In recent years there has been a tendency to produce, in addition to gasoline, light olefins by FCC operations, which are valuable raw materials for various chemical processes. These operations have significant economic advantages, particularly with respect to oil refineries that are highly integrated with petrochemical production facilities. Further FCC operations are based on cracking the feed oil at high temperature and short contact time and using a catalyst mixture of specific base cracking catalyst and an additive containing a shape-selective zeolite, also known as high severity fluidized catalytic cracking (“HS-FCC”). Features of this process include a downflow reactor, high reaction temperature, short contact time, and high catalyst to oil ratio.

Specific catalyst formulations and/or additives are employed to enhance the octane number of gasoline fractions, or to increase yield of light olefins such as propylene. Worldwide demand for propylene is growing continuously, and greater than 30% of propylene produced in the world is attained from FCC processes. Therefore, there are ongoing needs for catalyst development.

The design of catalysts is challenging, and when one is interested in a specific reaction or specific reactions, multiple options are presented. Every user has its own selection criteria for the catalyst(s) used. These include, e.g., the nature of the feedstock, the objective, the desired yield, and so forth. The factors that are at issue with respect to selection of a catalyst generally require the user to test multiple options before proceeding with the catalyst selected. These testing procedures are expensive and require a great deal of time.

A widely used test method for FCC catalysts include the standardized fixed bed microactivity test (MAT), as defined by ASTM-D3907. In such a test, a relatively small sample of a few grams of catalyst is loaded into a fixed bed and a vapor feed is passed through the bed. Another available option for small scale reactor testing is marketed by Kayser Technology, Inc. (Pasadena, TX, USA) under the trademark name ACE Technology®. Pilot plant designs include once-through and circulating designs, and a common unit is the DCR™ pilot plant (Grace Catalysts Technologies, Columbia, MD, USA).

Despite these recognized ways to test FCC catalysts, there remains a need in the art for improved testing processes and apparatuses. It is in regard to these and other problems in the art that the present disclosure is directed to provide a technical solution for improved processes and apparatuses for testing FCC catalysts.

### SUMMARY OF THE DISCLOSURE

A process to assess efficacy of at least one property of regenerated fluid catalytic cracking (FCC) catalyst is provided. The process comprises: withdrawing a minor portion of the regenerated FCC catalyst from a main FCC zone for testing; introducing the withdrawn regenerated FCC catalyst to a testing zone including at least one test reactor; introducing at least one test feedstock to the at least one test reactor containing the regenerated FCC catalyst for reaction to produce effluent test hydrocarbons and used test catalyst; and assessing at least one property of effluent test hydrocarbons, used test catalyst, or both effluent test hydrocarbons and used test catalyst.

In certain embodiments, the minor portion of the regenerated FCC catalyst comprises 0.0001-2, 0.0001-1, 0.0001-0.5, 0.0005-2, 0.0005-1 or 0.0005-0.5 wt % of regenerated. FCC catalyst from the retain FCC zone. In certain embodiments, the retain FCC zone includes at least a regeneration zone and a main FCC reactor, regenerated FCC catalyst is passed from the regeneration zone to the main FCC reactor, and the minor portion of regenerated FCC catalyst that is withdrawn for testing is from the regeneration zone. The regenerated. FCC catalyst may be withdrawn at predefined intervals, continuously, continuously with intermittent interruption, continuously with interruption or at predefined intervals. In certain embodiments, the testing zone comprises a scaled down test reactor that is 1-25, 1-20, 1-15, or 1-10% of the size of the main FCC reactor.

In some embodiments, the at least one test feedstock comprises a portion of a hydrocarbon feedstock used in the main FCC zone, the test reactor operates with parameters that differ from corresponding operational parameters of the main FCC zone, and the parameters include catalyst to oil ratio, temperature, residence time, injection location of the one or more of the at least one hydrocarbon feedstocks and/or withdrawn regenerated FCC catalyst, or combinations of the parameters. In some embodiments, the at least one test feedstock comprises a hydrocarbon feedstock that is different than that used in the main FCC zone obtained from distillation, delayed coking, hydrocracking or hydrotreating units. In some embodiments, an additional catalyst or catalyst additive is introduced to the test reactor.

In certain embodiments, the testing zone comprises a plurality of test reactors in parallel, and each test reactor receives regenerated FCC catalyst and one or more of the at least one test feedstocks. In some embodiments, the plurality of test reactors includes a first reactor and a second reactor, wherein during reaction the first reactor operates at a first set of parameters, the second reactor operates at a second set of parameters, and the first set of parameters and the second set of parameters contain at least one parameter that is different. In some embodiments, the at least one parameter of the first set of parameters and the second set of parameters that are different include one or more of catalyst to oil ratio, temperature, residence time and injection location of the one or more of the at least one hydrocarbon feedstocks. In some embodiments, the at least one test feedstock comprises at least two test feedstocks including a first test feedstock and a second test feedstock which are different from one another, the plurality of test reactors includes a first reactor and a second reactor, and the first test feedstock is introduced into the first actor and the second test feedstock is introduced into the second reactor. In some embodiments, the first test feedstock comprises a hydrocarbon mixture corresponding to that used in the main FCC zone from which the regenerated FCC catalyst for testing is obtained, and the second test feedstock comprises a hydrocarbon mixture that differs from that used in the main FCC zone from which the regenerated FCC catalyst for testing is obtained. In certain embodiments, the testing zone comprises a plurality of test reactors in parallel, wherein at least one test reactor receives regenerated FCC catalyst and one or more of the at least one test feedstocks, and at least one test reactor receives fresh FCC catalyst and optionally fresh FCC catalyst additives.

In certain embodiments, the regenerated FCC catalyst for testing is heated, and the heated regenerated FCC catalyst for testing heats the at least one test feedstock in the at least one test reactor. In embodiments herein, the test reactor operates at a temperature in the range of about 300-700° C. In embodiments herein, the test reactor operates with a ratio of the regenerated FCC catalyst for testing to the at least one hydrocarbon feedstock of 1:1 to 60:1, 1:1 to 40:1, 1:1 to 30:1, 3:1 to 60:1, 3:1 to 40:1, 3:1 to 30:1, 8:1 to 60:1, 8:1 to 40:1 or 8:1 to 30:1 on a weight basis. In certain embodiments, the testing zone further comprises a solid-gas-liquid separation system.

In certain embodiments, at least one property of effluent test hydrocarbons is assessed, and the at least one property of effluent test hydrocarbons is selected from the group consisting of heteroatom content, simulated distillation data, hydrogen content, density, PONA content, NONA, relative yield fractions, light olefin content, hydrogen sulfide content, Reid vapor pressure (RVP), octane number, cetane index, smoke point and pour point. In certain embodiments, at least one property of used test catalyst is assessed, and the at least one property of used test catalyst is selected from the group consisting of coke content, heteroatom content and mechanical integrity.

Any combinations of the various embodiments and implementations disclosed herein can be used. These and other aspects and features can be appreciated from the following description of certain embodiments and the accompanying drawings and claims.

### BRIEF DESCRIPTION OF DRAWINGS

The following detailed description of specific embodiments of the present disclosure can be best understood when read in conjunction with the following drawings, where like structure is indicated with like reference numerals and in which:

FIG. 1 shows a general embodiment of a system and process herein using a testing zone associated with a main FCC zone.

FIG. 2 shows another embodiment of a system and process herein using multiple testing zones associated with a main FCC zone.

FIG. 3 is a schematic representation of an FCC operation with a riser FCC zone including an integrated testing zone.

FIG. 4 is a schematic representation of an FCC operation with a downflow FCC zone including an integrated testing zone.

FIG. 5 is a schematic representation of a testing zone integrating a riser test reactor.

FIG. 6 is a schematic representation of a testing zone integrating a downflow test reactor.

### DETAILED DESCRIPTION

The present disclosure provides systems and methods for assessing FCC catalysts, in particular, FCC catalysts obtained from an operational FCC unit to assess viability with different FCC feed, additional catalyst additives, and/or one or more other FCC operational parameters. The systems and methods provide the operator the ability to test the FCC catalysts under real time conditions. A testing zone that can assess the FCC catalyst from an operational FCC unit is installed adjacent to a commercial FCC unit and receives a sample of the FCC catalyst, which is identical to one used in the commercial FCC unit. The methods herein differ from those using a typical pilot plant as it can duplicate the actual catalyst in real time from a commercial FCC unit, which is not practiced in typical pilot plants. The methods herein permit vast reductions in the amount of time necessary to assess the viability of the FCC catalyst from the operational FCC unit under different conditions, for different feeds, and/or with additional/different catalyst additives, resulting in vast reductions in the cost of this aspect of FCC operations.

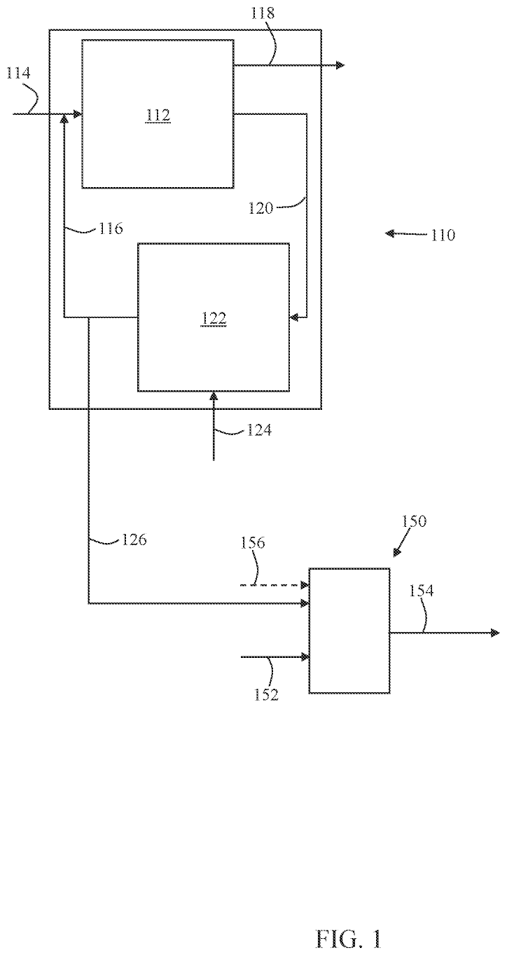

FIG. 1 shows a general embodiment of a system for carrying out a method for testing FCC catalysts herein. The system generally includes a main FCC zone 110 and a testing zone 150 (or one or more testing zones) to test a sample of the actual regenerated catalyst used in the main FCC zone 110, which can be a commercial, operational FCC zone. In general, the FCC zone 110 includes: a main FCC reactor 112 that receives a feedstock 114 and regenerated solid cracking catalyst particles 116, and discharges FCC effluent 118 and spent or partially spent catalyst 120 (typically through a cyclone or other solids separation apparatus to remove catalyst particles, not shown) and a catalyst regeneration zone 122 that receives the spent or partially spent catalyst 120 and an oxygen-containing gas 124, such as pure oxygen or air, and discharges the regenerated solid cracking catalyst particles 116 that are passed to the main FCC reactor 112. The FCC effluents 118 are typically fractioned in a product recovery section into light olefins, gasoline, light cycle oil and heavy cycle oil (not shown).

A portion 126 of the heated regenerated (or partially regenerated) solid cracking catalyst particles 116 are directed to the testing zone 150, where they are used for reaction of a test feedstock 152 to produce test effluents 154. In certain optional embodiments, the testing zone 150 is operable to receive additional catalyst 156, which can be an additional FCC catalyst or an FCC catalyst additive. In certain embodiments, in which fresh catalyst is added, the catalyst can be preheated, or the feed heated, to maintain a requisite temperature for the test reactor. The portion 126 is a minor portion of the total amount of regenerated solid cracking catalyst particles 116 from the regenerator 122 of the main FCC zone 110 for instance, about 0.0001-2, 0.0001-1, 0.0001-0.5, 0.0005-2, 0.0005-1 or 0.0005-0.5 wt %. In certain embodiments, the minor portion 126 of regenerated solid cracking catalyst particles and the test feedstock 152 are introduced to a reactor of the testing zone concurrently.

During operation, the portion 126 of the regenerated solid cracking catalyst particles 116 are directed from the main FCC zone 110 to the testing zone 150. In certain embodiments, the portion 126 can be withdrawn continuously with the flow of regenerated catalyst from the regeneration zone to the reactor. In certain embodiments, the portion 126 can be withdrawn continuously with the flow of regenerated catalyst from the regeneration zone to the reactor, with intermittent interruption and/or with interruption at predetermined intervals for a predetermined period of time. In certain embodiments, the portion 126 can be withdrawn at intervals such at predefined intervals for example in the range of about 0.01-100, 0.01-30, 0.01-14, 0.01-7, 0.04-1, 0.04-100, 0.04-30, 0.04-14, 0.04-7, 0.04-1, 0.5-100, 0.5-30, 0.5-14, 0.5-7, 0.5-1 days (for instance, about every 0.25, 0.5, 1, 2, 6, 12, 24, 48, 72, 96, 120, 144 or 168 hours, or about every 0.5, 1, 1.5, 2, 3, 4, 5, 6, 7, 14 or 30, 60 or 90 days). In certain embodiments, the portion 126 can be withdrawn intermittently such as at a time desired by the operator as needed to pass to the testing zone 150, for example, when an intentional or unintentional change in a feedstock or parameter of the main FCC zone 110 is encountered. In embodiments in which withdrawing of the portion 126 is at intervals or intermittently, the withdrawal can occur for a continuous period of time effective to obtain a portion 126 that is of sufficient quantity of regenerated catalyst to carry out the assessment in the testing zone 150, for example, in the range of about 1-3600, 5-3600, 10-3600, 30-3600, 60-3600, 1-1800, 5-1800, 10-1800, 30-1800, 60-1800, 1-900, 5-900, 10-900, 30-900 or 60-900 seconds.

In certain embodiments, the temperature of the portion 126 may be maintained at its temperature at the time of withdrawal, so that the temperature for integration in the testing zone 150 is the same as or approximately equivalent to the temperature of regenerated catalyst passed from the regenerator 122 to the main FCC reactor 112 in the main FCC zone 110. In certain embodiments the temperature of the portion 126 may drop to less than the temperature at the time of withdrawal, and integration in the testing zone 150 may include pre-heating the sample to a temperature the same as or approximately equivalent to the temperature at the time of withdrawal.

The effluents 154 are analyzed using known testing methods to assess properties about the catalyst, including the efficacy of the regenerated solid cracking catalyst that are actively in use in the commercial FCC operation. For example, efficacy can be assessed by determining yield and compositional information regarding the test effluents. This can be accomplished using a feed that is the same or different than the main FCC zone feed, and/or by running the testing zone 150 with variation in one or more parameters, such as one or more of catalyst to oil ratio, catalyst additives, temperature, residence time, feedstock injection location, and/or others.

In certain embodiments, the test effluents 154 are tested as a total effluent stream. In certain embodiments, the operator may carry out testing and/or analysis of the total test effluent stream 154 in terms of quantity, for example, yield relative to the test feedstock 152. In certain embodiments, the operator may test the total effluent stream 154 in in terms of quality, for example: heteroatom content, simulated distillation data, hydrogen content, density, “paraffins, olefins, naphthenes and aromatics” (PONA) content, “paraffins (n-paraffins), iso-paraffins, olefins, naphthenes and aromatics” (POONA) content, and/or others.

In certain embodiments, the test effluents 154 are tested after separation into a gas and liquid fraction or separated based on a predetermined boiling point. In certain embodiments, the operator may separate the effluents into a liquid and gas fraction and test these fractions individually in terms of quantity, for example, relative yield of each fraction. In certain embodiments, the operator may test the total liquid effluent in terms of quality, for example, for one or more of heteroatom content, simulated distillation data, hydrogen content, density, PONA, PIONA and/or others. In certain embodiments, the operator may test the total gas effluent in terms of quality, for example, light olefin content, hydrogen sulfide content, Reid vapor pressure (RVP), and/or others.

The testing zone 150 may include a separation device (not shown) to separate test effluents 154. In certain embodiments, such a separation device may operate to separate the test effluents 154 into fractions that are equivalent to the product recovery section of the main FCC zone 110, corresponding to the product recovery section of the main FCC unit products, for example, light olefins, gasoline, light cycle oil and heavy cycle oil; testing may be carried out on one or more of these fractions. In certain embodiments, the operator may test the individual fractions from the testing zone 150 in terms of relative quantity, for example, yields of individual fractions in the total effluent. In certain embodiments, the operator may test the one or more of the individual fractions from the testing zone 150 in terms of quality, for example, octane number (for instance of a FCC gasoline fraction), content of heteroatoms in any of the fractions, simulated distillation data for any of the fractions, density of any of the fractions, cetane index (for instances of a light cycle oil fraction), smoke point, pour point, and/or others. In certain embodiments, such a separation device may operate to separate the test effluents 154 into other fractions such as, for example, based on a flash separation to separate into a liquid and gas faction, or one or more other cut points; testing may be carried out on one or more of these fractions. In some embodiments, a separation device may include a solid-gas-liquid separator to separate catalyst, gases and total liquid products.

In certain embodiments, the used test catalyst from the testing zone 150, for example, in the absence of regeneration, may be tested and/or analyzed to assess behavior with the selected test feed 152 and/or operating conditions and/or optional additional of catalyst and/or catalyst additive materials 156. For example, levels of coke and/or heteroatom content can be determined. In addition, mechanical integrity including shock resistance and/or attrition indices may be assessed with respect to the used test catalyst.

In certain embodiments, the test feedstock 152 comprises, consists essentially of, or consists of, a hydrocarbon stream identical to the FCC feedstock 114, and the catalyst passed to the testing zone 150 comprises, consists essentially of, or consists of, the portion 126 of the heated regenerated solid cracking catalyst particles. In such embodiments, in which a test feedstock and the catalyst used in the testing zone 150 are the same as the main FCC zone 110, one or more other parameters can be varied for testing purposes. For example, conditions in the test reactor can vary from the main reactor system for testing, such as one or more of catalyst to oil ratio, temperature, residence time, feedstock injection location, and others. In certain embodiments, in which a test feedstock and the catalyst used in the testing zone 150 are the same as the main FCC zone 110, an additional catalytic material 156 is included, which can be a type of catalyst material different than the portion 126 and/or FCC catalyst additives. In certain embodiments, the additional catalytic material 156 is heated so that the temperature is approximately equivalent to the temperature of the portion 126.

In some embodiments, the test feedstock 152 comprises a hydrocarbon stream identical to the FCC feedstock 114, and the catalyst passed to the testing zone 150 comprises the portion 126 of the heated regenerated solid cracking catalyst particles, wherein the catalyst to oil ratio is varied as compared to the main FCC zone 110. Test effluents 154 (as a whole, after partial separation, or after separation into fractions corresponding to the product recovery section of the main FCC zone products) can be analyzed to ascertain variations in yield and/or composition compared to the FCC effluents 118 due to variations in the catalyst to oil ratio.

In further embodiments, the test feedstock 152 comprises a hydrocarbon stream identical to the FCC feedstock 114, and the catalyst passed to the testing zone 150 comprises the portion 126 of the heated regenerated solid cracking catalyst particles, wherein an additional catalyst additive is provided (e.g., as represented by line 156) which is not used in the main FCC zone 110. The catalyst additive may be heated to an approximately equivalent temperature as the portion 126 of the heated regenerated solid cracking catalyst particles. Test effluents 154 (as a whole, after partial separation, or after separation into fractions corresponding to the product recovery section of the main FCC zone products) can be analyzed to ascertain variations in yield and/or composition compared to the FCC effluents 118 due to the inclusion of the catalyst additive.

In certain embodiments, the test feedstock 152 comprises a hydrocarbon stream identical to the FCC feedstock 114, and the catalyst passed to the testing zone 150 comprises the portion 126 of the heated regenerated solid cracking catalyst particles, wherein a catalyst additive is provided (e.g., as represented by line 156). The catalyst additive may be heated to an approximately equivalent temperature as the portion 126 of the heated regenerated solid cracking catalyst particles. Test effluents 154 (as a whole, after partial separation, or after separation into fractions corresponding to the product recovery section of the main FCC zone products) can be analyzed to ascertain variations in yield and/or composition compared to the FCC effluents 118 due to an increased amount of the catalyst additive.

In some embodiments, the test feedstock 152 comprises a hydrocarbon stream identical to the FCC feedstock 114, and the catalyst passed to the testing zone 150 comprises the portion 126 of the heated regenerated solid cracking catalyst particles, wherein the temperature of the test reaction is different than that in the main FCC zone 110. Temperature may be varied by heating or cooling the catalyst prior to introduction in the testing zone 150. Test effluents 154 (as a whole, after partial separation, or after separation into fractions corresponding to the product recovery section of the main FCC zone products) can be analyzed to ascertain variations in yield and/or composition compared to the FCC effluents 118 due to a decrease or increase in reaction temperature.

In certain embodiments, the test feedstock 152 comprises a hydrocarbon stream identical to the FCC feedstock 114, and the catalyst passed to the testing zone 150 comprises the portion 126 of the heated regenerated solid cracking catalyst particles, wherein the residence time in the test rector is different than that in the main FCC zone 110. Test effluents 154 (as a whole, after partial separation, or after separation into fractions corresponding to the product recovery section of the main FCC zone products) can be analyzed to ascertain variations in yield and/or composition compared to the FCC effluents 118 due to a decrease or increase in residence time.

In some embodiments, the test feedstock 152 comprises a hydrocarbon stream identical to the FCC feedstock 114, and the catalyst passed to the testing zone 150 comprises the portion 126 of the heated regenerated solid cracking catalyst particles, wherein the injection location (that is, the relative location along the riser or downflow reactor in which catalyst and/or feed are injected) is different than that in the main FCC zone HO. Test effluents 154 (as a whole, after partial separation, or after separation into fractions corresponding to the product recovery section of the main FCC zone products) can be analyzed to ascertain variations in yield and/or composition compared to the FCC effluents 118 due to a change in the injection location.

In certain embodiments, the test feedstock 152 comprises a hydrocarbon stream different than the FCC feedstock 114, and the catalyst passed to the testing zone 150 comprises, consists essentially of, or consists of, the portion 126 of the heated regenerated solid cracking catalyst particles. In such embodiments, the testing zone 150 is employed to evaluate the use of the regenerated catalyst from the main FCC zone for cracking the different type of hydrocarbon stream as the test feedstock, and the conditions in the test reactor can be approximately equivalent to, or different from, those in main FCC zone, including catalyst to oil ratio, temperature, residence time, feedstock injection location, and others. In certain embodiments, a test feedstock that differs from the main FCC feedstream comprises, consists essentially of, or consists of an approximately equivalent fraction from a different original source (for instance, from a different initial crude oil). In certain embodiments, a test feedstock that differs from the main FCC feedstream comprises, consists essentially of, or consists of a different fraction from the original source, for instance, a similar fraction from the same initial source (such as the same initial crude oil source) as the feedstream to the main FCC zone with a variation in the upper and/or lower temperature range. In certain embodiments, a test feedstock that differs from the main FCC feedstream comprises, consists essentially of, or consists of a portion of the fraction of the feedstream to the main FCC zone, for instance, where the main FCC zone feed is vacuum gas oil, the test feedstocks can comprise light vacuum gas oil or heavy vacuum gas oil. In certain embodiments, a test feedstock that differs from the main FCC feedstream comprises, consists essentially of, or consists of a feedstream that has a different relative reactivity. In certain embodiments, a test feedstock that differs from the main FCC feedstream comprises, consists essentially of, or consists of a feed of lighter hydrocarbons, for instance in middle distillates when a main FCC feed is vacuum gas oil. In certain embodiments, a test feedstock that differs from the main FCC feedstream comprises, consists essentially of, or consists of a feed of heavier hydrocarbons, for instance vacuum residue when a main FCC feed is vacuum gas oil. In certain embodiments, a test feedstock may comprise, consists essentially of, or consists of naphtha range hydrocarbons. In certain embodiments, a test feedstock may comprise, consists essentially of, or consists of middle distillate range hydrocarbons. In certain embodiments, a test feedstock may comprise, consists essentially of, or consists of atmospheric reside hydrocarbons.

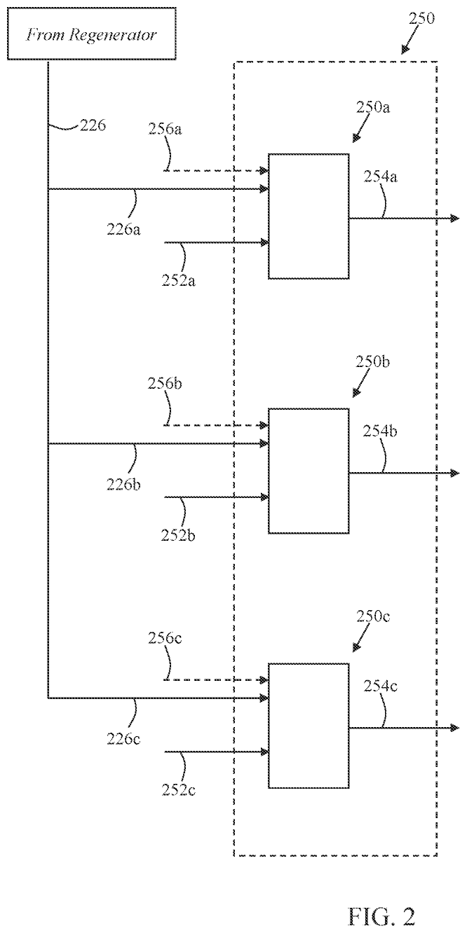

FIG. 2 depicts another arrangement, a testing zone 250 that includes a plurality of test reactor sub-systems 250a, 250b and 250c which are arranged in parallel (although as few as two and as many as necessary, for instance, 10, 20 or more, can be provided). A portion 226 of the heated regenerated solid cracking catalyst particles from a regenerator of a main FCC zone (not shown in FIG. 2) is divided into plural sub-portions 226a, 226b and 226c, which are passed to the test reactor sub-systems 250a, 250b and 250c respectively. Each of the test reactor sub-systems 250a, 250b and 250c receives a test feedstock, 252a, 252b and 252c respectively. Optionally, each of the test reactor sub-systems 250a, 250b and 250c receives an additional catalyst or catalyst additive, 256a, 256b and 256c respectively. The test reactor sub-systems 250a, 250b and 250c each discharge a test effluent, 254a, 254b and 254c respectively.

In a testing zone 250, an operator may simultaneously test the same heated regenerated solid cracking catalyst as is in us a main FCC zone. In certain embodiments, the test feedstocks 252a, 252b and 252c are approximately equivalent to the feedstream used in the main FCC zone, with variations in one or more operating parameters in the sub-systems 250a, 250b and 250c, In certain embodiments, the test feedstocks 252a, 252b and 252c are each approximately equivalent feeds and are different from the feedstream to the main FCC zone, with a variation in one or more operating parameters between each of the sub-systems 250a, 250b and 250c. In certain embodiments, a plurality of different test feeds as described hereinabove can be tested under the approximately equivalent conditions in each of the different test sub-systems. In certain embodiments, one of the test feedstocks to the test sub-systems (for instance 252a) is the same as the feedstream used in the main FCC zone, and the other test feedstocks (for instance 252b and 252c) are different test feeds as described hereinabove.

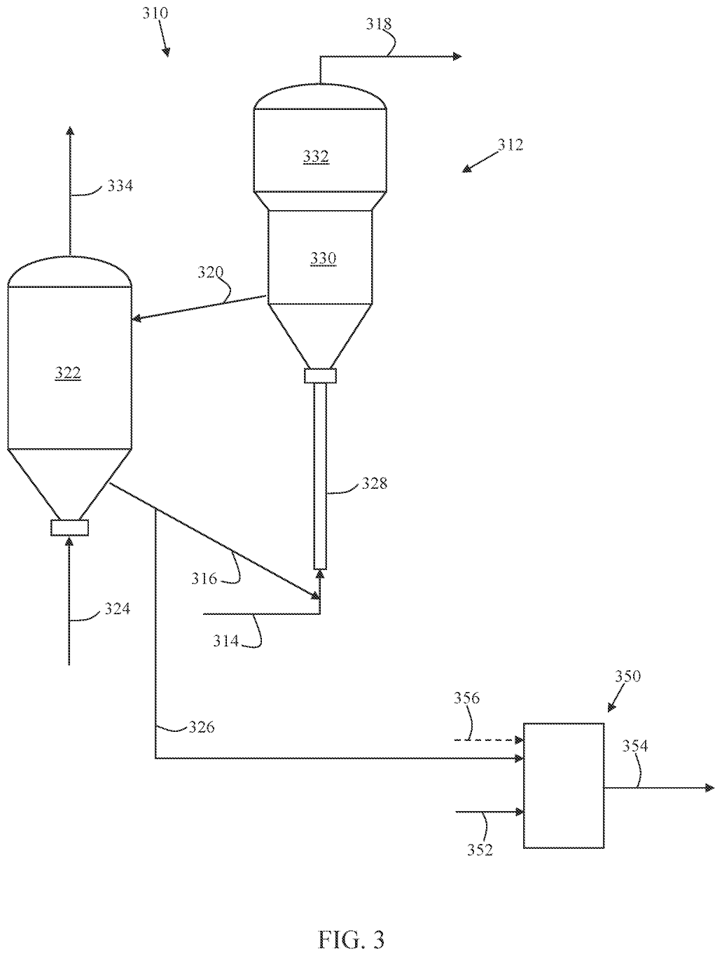

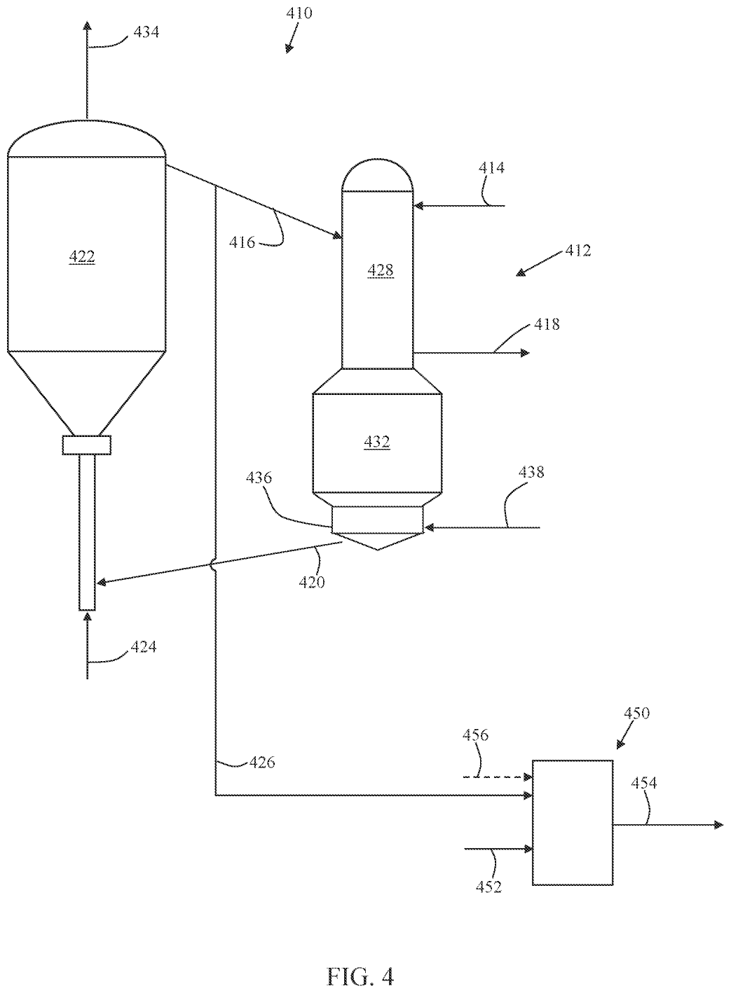

FIGS. 3 and 4 are schematic diagrams of an integrated testing examples of FCC reactors that lay have integrated therein a testing zone. It will be appreciated that these diagrams are exemplary, and the testing zone can be integrated in other types of FCC reactors. There are many commercially available FCC systems, including those primarily for gasoline production and those for propylene production which may benefit from the systems and methods for testing of FCC catalysts described herein to test a sample of the actual regenerated catalyst used.

In certain embodiments, an FCC zone configured with a riser reactor is provided and is schematically depicted in FIG. 3, and a testing zone 350 is integrated to assess the FCC catalyst from an operational FCC zone for different feedstocks and/or different conditions and/or with additional FCC catalyst or catalyst additive. A riser FCC zone 310 includes a reactor/separator 312 having a riser reactor portion 328, a stripping zone 330 and a separation zone 332; and a regeneration zone 322 for regenerating spent catalyst. A feedstock 314 is introduced to the riser reactor, in certain embodiments accompanied by steam or other suitable gas for atomization and lift of the feed (not shown). A feedstock 314 is admixed and intimately contacted with an effective quantity of heated and regenerated solid cracking catalyst particles which are conveyed via a conduit 316 from the regeneration zone 322. Optionally (not shown) additional heated fresh catalyst can be introduced in the system, for example via a separate inlet to the riser reactor 328 or combined with the regenerated catalyst in conduit 316. The feed mixture and the cracking catalyst are contacted under conditions to form a suspension that is introduced into the riser reactor 328. In a continuous process, the mixture of cracking catalyst and hydrocarbon feedstock proceed upward through the riser reactor 328 into the stripping zone 330. In the riser reactor 328 and the stripping zone 330, the hot cracking catalyst particles catalytically crack relatively large hydrocarbon molecules by carbon-carbon bond cleavage. It is understood that cracking reactions typically continues to some extent in the stripping zone.

During the reaction, as is typical in FCC operations, the cracking catalysts become coked, and hence access to the active catalytic sites is limited or nonexistent. Reaction products are separated from the coked catalyst using any suitable configuration known in FCC units, generally referred to as the separation zone 332 in an FCC zone 310, for instance, located at the top of the reactor/separator 312 above the stripping zone 330. For example, steam may be used in the stripping zone 330. The separation zone can include any suitable apparatus known to those of ordinary skill in the art such as, for example, cyclones. Other configurations are also known and within the scope of the processes and systems herein.

The reaction product is withdrawn through conduit 318. Catalyst particles containing coke deposits from fluid cracking of the hydrocarbon feedstock pass through a conduit 320 to the regeneration zone 322. In the regeneration zone 322, the coked catalyst comes into contact with a stream of oxygen-containing gas, such as pure oxygen or air, which enters the regeneration zone 322 via a conduit 324. The regeneration zone 322 is operated in a configuration and under conditions that are known in typical FCC operations. For instance, the regeneration zone 322 can operate as a fluidized bed to produce regeneration off-gas comprising combustion products produced through combustion of the coke deposits which is discharged through a conduit 334. Combustion of the coke deposits in the regeneration zone 322 may heat the regenerated FCC catalyst 316 to a temperature greater than or equal to the reaction temperature in the riser reactor 328. The hot regenerated catalyst is transferred from the regeneration zone 322 through the conduit 316 to the bottom portion of the riser reactor 328 for admixture with the hydrocarbon feedstock and noted above.

In one embodiment, the operating conditions for the reactor of a suitable riser FCC zone 310 that receives a typical feed, for instance, vacuum gas oil or other heavy hydrocarbon feed, include: a reaction temperature (° C.) of from about 300-700, 450-700, 500-700, 550-700, 600-700, 650-700, 300-650, 450-650; a contact time (in the reactor, seconds) of from about 0.1-10, 0.1-5, 0.1-2, 0.5-10, 0.5-5, 0.5-2, 1-10, 1-5 or 1-2; and a catalyst-to-feed ratio (based on weight) of about 1:1 to 60:1, 1:1 to 20:1, 1:1 to 15:1, 1:1 to 10:1, 3:1 to 60:1, 3:1 to 20:1, 3:1 to 15:1, 3:1 to 10:1; 8:1 to 60:1, 8:1 to 20:1, 8:1 to 15:1 or 8:1 to 10:1.

The testing zone 350 can be similar to that described with respect to FIG. 1 or 2 and can act upon different feeds and/or under different parameters as described herein. For instance, the testing zone 350 receives a portion 326 of the heated regenerated solid cracking catalyst from the regenerator 322 (for instance, such as via a bleed stream from conduit 316), a feedstream 352, and optional additional catalyst or catalyst additive 356; and discharges a test effluent 354.

In certain embodiments, an FCC zone configured with a downflow reactor is provided and is schematically depicted in FIG. 4. Downflow reactors permit higher catalyst to oil ratio, since lifting of solid catalyst particles by vaporized feed is not required, and this is particularly suitable for high severity FCC operation. A downflow FCC zone 410 includes a reactor/separator 412 having a downflow reactor 428 and a separation zone 432; and a regeneration zone 422 for regenerating spent catalyst. A feedstock 414 is introduced to the downflow reactor, and, in certain embodiments, is accompanied by steam or other suitable gas for atomization of the feed (not shown). An effective quantity of hot regenerated solid cracking catalyst particles from the regeneration zone 422 are conveyed to the top of the downflow reactor 428 and also transferred, for instance, through a downwardly directed conduit or pipe 416 that is commonly referred to as a transfer line or standpipe, to a withdrawal well or hopper (not shown) at the top of downflow reactor 428. Optionally, additional heated fresh catalyst (not shown) can be introduced in the system, for example, via a separate inlet to the downflow reactor 428 or combined with the regenerated catalyst in conduit 416. Hot catalyst flow is typically allowed to stabilize in order to be uniformly directed into the mix zone or the feed injection portion of the downflow reactor 428. The charge 414 is injected into a mixing zone through feed injection nozzles typically situated proximate to the point of introduction of the regenerated catalyst into downflow reactor 428. These multiple injection nozzles result in the thorough and uniform mixing of the hot catalyst and the charge 414 in the integrated process herein hydrotreated gas oil, optionally in combination with atmospheric gas oil such as heavy atmospheric gas oil. Once the charge contacts the hot catalyst, cracking reactions occur.

The reaction vapor of hydrocarbon cracked products, unreacted feed, and catalyst mixture quickly flows through the remainder of the downflow reactor 428 and into the rapid separation zone 432 at the bottom portion of the reactor/separator 412. Cracked and uncracked hydrocarbons are directed through a conduit or pipe 418 to a conventional product recovery section (not shown) that is known in the art to yield as fluid catalytic cracking products light olefins, gasoline and cycle oil. If necessary, for temperature control, a quench injection (not shown) can be provided near the bottom of the downflow reactor 428 immediately before the separation zone 432. This quench injection quickly reduces or stops the cracking reactions and can be utilized for controlling cracking severity to achieve the product slate.

The reaction temperature, for instance, the outlet temperature of the downflow reactor, can be controlled by opening and closing a catalyst slide valve (not shown) that controls the flow of hot regenerated catalyst from the regeneration zone 422 into the top of the downflow reactor 428. The heat required for the endothermic cracking reaction is supplied by the regenerated catalyst. By changing the flow rate of the hot regenerated catalyst, the operating severity or cracking conditions can be controlled to produce the desired product slate. A stripper 436 is also provided for separating oil from the catalyst, which is transferred to the regeneration zone 422. The catalyst from the separation zone 432 flows to the lower section of the stripper 436 that includes a catalyst stripping section into which a suitable stripping gas, such as steam, is introduced through streamline 438. The stripping section is typically provided with several baffles or structured packing (not shown) over which the downwardly flowing catalyst passes counter-currently to the flowing stripping gas. The upwardly flowing stripping gas, which is typically steam, is used to “strip” or remove any additional hydrocarbons that remain in the catalyst pores or between catalyst particles. The stripped and spent catalyst is transported via a conduit 420 to the regeneration zone 422, where it is transported through a lift riser of the regeneration zone 422 by lift forces from a combustion air stream 424. This spent catalyst, which can also be contacted with additional combustion air, undergoes controlled combustion of any accumulated coke. Flue gases are removed from the regenerator via conduit 434. In the regeneration zone 422, the heat produced from the combustion of the by-product coke is transferred to the catalyst raising the temperature required to provide heat for the endothermic cracking reaction in the downflow reactor 428. For instance, the regeneration zone 422 may be operated as a fluidized bed to produce regenerated FCC catalyst 416 and flue gas via conduit 434, which may include combustion products produced through combustion of the coke deposits. Combustion of the coke deposits in the regeneration zone 422 may heat the regenerated FCC catalyst 416 to a temperature greater than or equal to the reaction temperature in the downflow reactor 428. The regenerated FCC catalyst 416 is to be transferred from the regeneration zone 422 to the downflow reactor 428.

In one embodiment, the operating conditions for a suitable downflow FCC zone 410 that receives a typical feed, for instance, a vacuum gas oil or other heavy hydrocarbon feed, include: a reaction temperature (° C.) of from about 300-700, 450-700, 500-700, 550-700, 600-700, 650-700, 300-650, 450-650; a contact time (in the reactor, seconds) of from about 0.1-30, 0.1-10, 0.1-0.7, 0.2-30, 0.2-10 or 0.2-0.7; and a catalyst-to-feed ratio (based on weight) of about 1:1 to 60:1, 1:1 to 40:1, 1:1 to 30:1, 3:1 to 60:1, 3:1 to 40:1, 3:1 to 30:1, 8:1 to 60:1, 8:1 to 40:1 or 8:1 to 30:1.

The testing zone 450 can be similar to that which is described with respect to FIG. 1 or 2 and can act upon different feeds and/or under different parameters as described herein. For instance, the testing zone 450 receives a portion 426 of the heated regenerated solid cracking catalyst from the regenerator 422 (for instance via a bleed stream from conduit 416), a feedstream 452, and optional additional catalyst or catalyst additive 456; and discharges a test effluent 454.

The testing zones 150, 250, 350, 450 generally include one or more test reactors arranged in series and/or in parallel. The testing zone herein is dimensioned accordingly for testing purposes. In certain embodiments, the testing zone generally does not include a dedicated catalyst regenerator that is conventional with the main FCC zone. For example, each test reactor can contain one or more single or dual riser reactors or downflow reactors, which are scaled down relative to the riser reactors or downflow reactors of the main FCC zone. For instance, commercial riser FCC units generally include a main FCC reactor having risers with heights or lengths in the range of about 1,000-3,700 centimeters and diameters in the range of about 61-213 cm. Commercial downflow reactors are of similar dimensions. In contrast a test reactor of a testing zone herein may have a height or length in the range of about 10-950, 10-750, 10-560, 10-370, 10-250, 10-200, 10-150, 10-100, 35-950, 35-750, 35-560, 35-370, 35-250, 35-200, 35-150, 35-100, 100-950, 100-750, 100-560, 100-370, 100-250, 100-200, 100-150 centimeters and a diameter in the range of about 0.5-55, 0.5-45, 0.5-35, 0.5-25, 0.5-15, 0.5-10, 0.5-7, 2-55, 2-45, 2-35, 2-25, 2-15, 2-10 or 2-7 centimeters. In certain embodiments, a test reactor is generally 1-25, 1-20, 1-15, or 1-10% of the size of the main FCC reactor. For example: A test riser reactor or a test downflow reactor of the test system 150, 250, 350, 450 may have a height or length that is about 1-25, 1-20, 1-15, or 1-10% of the height or length of the main FCC riser or downflow reactor of the main FCC zone; and a test riser reactor or a test downflow reactor of the testing zone 150, 250, 350, 450 may have a diameter that is about 1-25, 1-20, 1-15, or 1-10% of the diameter of the riser reactor or downflow of the main FCC zone.

It is to be appreciated that the configuration of the test reactor in the testing zone may be the same or different than that used in the main FCC zone. In embodiments in which the main FCC zone includes a riser reactor, the testing zone may include one or more test riser reactors, one or more test downflow reactors, or a combination of one or more test riser reactors and one or more test downflow reactors. In embodiments in which the main FCC zone includes a downflow reactor, the testing zone may include one or more test downflow reactors, one or more test riser reactors, or a combination of one or more test downflow reactors and one or more test riser reactors.

Conditions in the testing zone test reactor can emulate those in commercial FCC units. For example, in embodiments in which the test reactor includes a scaled down riser reactor, conditions may include: a reaction temperature (° C.) of from about 300-700, 450-700, 500-700, 550-700, 600-700, 650-700, 300-650, 450-650; a contact time (in the reactor, seconds) of from about 0.1-10, 0.1-5, 0.1-2, 0.5-10, 0.5-5, 0.5-2, 1-10, 1-5 or 1-2; and a catalyst-to-feed ratio (based on weight) of about 1:1 to 60:1, 1:1 to 20:1, 1:1 to 15:1, 1:1 to 10:1, 3:1 to 60:1, 3:1 to 20:1, 3:1 to 15:1, 3:1 to 10:1; 8:1 to 60:1, 8:1 to 20:1, 8:1 to 15:1 or 8:1 to 10:1. In embodiments in which the test reactor includes a scaled down downflow reactor, conditions may include: A reaction temperature (° C.) of from about 300-700, 450-700, 500-700, 550-700, 600-700, 650-700, 300-650, 450-650; a contact time (in the reactor, seconds) of from about 0.1-30, 0.1-10, 0.1-0.7, 0.2-30, 0.2-10 or 0.2-0.7; and a catalyst-to-feed ratio (based on weight) of about 1:1 to 60:1, 1:1 to 40:1, 1:1 to 30:1, 3:1 to 60:1, 3:1 to 40:1, 3:1 to 30:1, 8:1 to 60:1, 8:1 to 40:1 or 8:1 to 30:1.

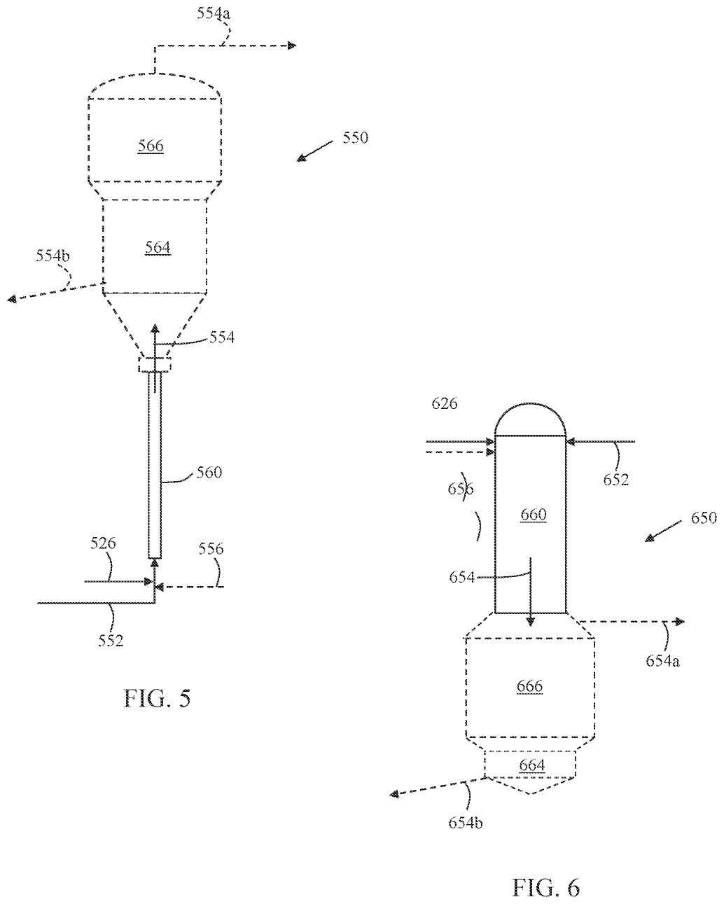

Referring to FIG. 5, an example of a testing zone 550 is provided that can operate as test system 150, 250, 350, 450 in the embodiments herein. The testing zone 550 generally including a riser test reactor 560 that receives test feedstock 552 and a portion 526 of regenerated catalyst from a main FCC zone, and discharges test effluents 554, which comprises reaction products and any unreacted hydrocarbons, and spent or partially catalyst. The test feedstock can be the same or different as the main FCC feed, as described herein. In certain embodiments, an optional additional catalyst and/or catalyst additive 556 is introduced to the riser test reactor 560 (shown in dashed lines), as described herein.

In certain embodiments, test effluents 554 can be subjected to the aforementioned testing procedures to assess quantitative or qualitative characteristics about the reaction products and/or the catalyst. If necessary, all or a portion of the test effluents 554 may be separated in another process (not shown) in conjunction with testing to assess quantitative or qualitative characteristics about the reaction products and/or the catalyst. In certain embodiments, as shown in dashed lines in FIG. 5, the testing zone 550 may include a test system stripping zone 564 and a test system separation zone 566. In the test system stripping zone 564 reaction products are separated from the coked catalyst using any suitable configuration known in FCC units, for example, by steam stripping. The test system separation zone 566 can include any suitable apparatus known to those of ordinary skill in the art such as, for example, cyclones; however, other configurations are also known and within the scope of the processes and systems herein. In embodiments in which the stripping zone 564 and the separation zone 566 are employed, cracked reaction products are discharged as a test effluent stream 554a, and spent catalyst 554b is discharged, either or both of which may be subjected to testing as described herein.

Referring to FIG. 6, an example of a testing zone 650 is provided that can operate as test system 150, 250, 350, 450 in the embodiments herein. The testing zone 650 generally including a downflow test reactor 660 that receives test feedstock 652 and a portion 626 of regenerated catalyst from a main FCC zone, and discharges test effluents 654, which comprises reaction products and any unreacted hydrocarbons, and spent or partially catalyst. The test feedstock can be the same or different as the main FCC feed, as described herein. In certain embodiments, an optional additional catalyst and/or catalyst additive 656 is introduced to the downflow test reactor 660 (shown in dashed lines), as described herein.

In certain embodiments, test effluents 654 can be subjected to the aforementioned testing procedures to assess quantitative or qualitative characteristics about the reaction products and/or the catalyst. If necessary, all or a portion of the test effluents 654 may be separated in another process (not shown) in conjunction with testing to assess quantitative or qualitative characteristics about the reaction products and/or the catalyst. In certain embodiments, as shown by dashed lines in FIG. 6, the testing zone 650 may include a test system separation zone 666 and a test system stripping zone 664, and reaction products and unreacted hydrocarbons are separated from the coked catalyst using any suitable configuration known in FCC units, for example, by steam stripping and cyclonic separation, or other configurations are also known and within the scope of the processes and systems herein, and discharge a test effluent stream 654a, and spent catalyst 654b, either or both of which may be subjected to testing as described herein.

The FCC catalysts used in the main FCC process described herein can be conventionally known or future developed catalysts used in FCC processes, such as zeolites, silica-alumina, carbon monoxide burning promoter additives, bottoms cracking additives, light olefin-producing additives and any other catalyst additives routinely used in the fluid catalytic cracking process. In certain embodiments, suitable cracking zeolites in the FCC process include zeolites Y, REY, USY, and RE-USY. In certain embodiments, one or more shape-selective catalyst additives can be employed, such as ZSM-5 zeolite crystal or other pentasil type catalyst structure used in FCC processes to produce light olefins and increase FCC gasoline octane; shape-selective catalyst additives can be mixed with a primary FCC catalyst. In the processes and systems herein, these catalysts, after regeneration, are passed to the testing zone including at least one test reactor.

In certain embodiments of the processes and systems herein, the testing zone may receive an additional (fresh) quantity of any of the herein disclosed conventionally known catalysts and/or catalyst additives routinely used in the fluid catalytic cracking process, or future developed catalysts. This additional quantity may be preheated as described herein prior to introduction in the testing zone including at least one test reactor. This additional quantity may be introduced in the testing zone combined with the portion of regenerated from the main FCC regenerator described herein. In certain embodiments, for example, those in which multiple test reactors are provided in parallel, one or more test reactors may receive a portion of regenerated from the main FCC regenerator, and one or more other test reactors may receive a portion of new catalyst.

In the FCC operations herein, the feedstock to the main FCC zone and the test reactor may be the same or different, and may be derived from an initial source that is one or more of crude oil, condensates, synthetic crude oil, bitumen, oil sand, shale oil, coal liquid, plastic pyrolysis oil and/or bio-mass derived oils. The feedstocks to the main FCC zone and the test reactor can be obtained from distillation, delayed coking, hydrocracking or hydrotreating units. For example, feedstock to the test reactor can be naphtha, diesel or heavy oils. Heavy oils can be any hydrocarbon typically having a nominal boiling point above about 350, 360, 370, 380, 390 or 400° C. For example, heavy oils can include one or more of atmospheric gas oil, vacuum gas oil, atmospheric residue, deasphalted oil obtained from a solvent deasphalting process, demetallized oil, light or heavy coker gas oil obtained from a coker process, gas oil obtained from a visbreaking process, and combinations comprising at least one of the foregoing heavy oils. Heavy oils can be treated or untreated prior to use as the feedstock to the main FCC zone and/or the test reactor. In certain embodiments, all or portions of FCC effluents, such as light and/or heavy cycle oil, are recycled with a main feedstock to the main FCC zone. In certain embodiments, all or portions of FCC effluents such as light and/or heavy cycle oil are used as feed to the test reactor. In certain embodiments, the main FCC zone is operated as a residue FCC zone, for example, in which the feedstock is atmospheric and/or vacuum residue.

It will also be understood that the test reactors can be adapted to simulate standard conditions of a commercial reactor, such as a start-up mode, steady-state operation, a shut-down mode, emergency modes, and so forth.

In operation, it is well known that feedstock to commercial FCC zones can include recycled materials. Further, additives can be included in the feedstock of the test reactors, so as to simulate products such as, but not being limited to, effluents from different refinery units such as hydrocrackers, hydrotreaters, coking units, steam cracking units, or others. In addition, heavier hydrocarbon fractions, such as a heavier portion of a vacuum gas oil fraction, can be added to ascertain the impact on stability and selectivity of the catalyst.

For the purpose of the simplified schematic illustrations and descriptions herein, accompanying components that are conventional in FCC zones including the numerous valves, temperature sensors, electronic controllers, air supplies, catalyst hoppers, cyclones, flue gas handling the like are not shown.

As used herein, the term “stream” (and variations of this term, such as hydrocarbon stream, feedstream, product stream, and the like) may include one or more of various hydrocarbon compounds, such as straight chain, branched or cyclical alkanes, alkenes, alkadienes, alkynes, alkylaromatics, alkenyl aromatics, condensed and non-condensed di-, tri- and tetra-aromatics, and gases such as hydrogen and methane, C2+ hydrocarbons and further may include various impurities.

The term “zone” refers to an area including one or more pieces of equipment, or one or more sub-zones. Equipment may include one or more reactors or reactor vessels including risers or downers, cyclones, catalyst regenerator, catalyst hoppers, heaters, heat exchangers, pipes, pumps, compressors, controllers, and product separation and/or purification apparatus. Additionally, equipment, such as reactor, dryer, or vessels, further may be included in one or more zones.

The term “weight percent” “wt %” refers to a relative value at conditions of 1 atmosphere pressure and 15° C., unless otherwise specified.

The phrase “a major portion” with respect to a particular stream or plural streams means at least about 50 wt % and up to 100 wt %, or the same values of another specified unit.

The phrase “a significant portion” with respect to a particular stream or plural streams means at least about 75 wt % and up to 100 wt %, or the same values of another specified unit.

The phrase “a substantial portion” with respect to a particular stream or plural streams means at least about 90, 95, 98 or 99 wt % and up to 100 wt %, or the same values of another specified unit.

The phrase “a minor portion” with respect to a particular stream or plural streams means from about 1, 2, 4 or 10 wt %, up to about 20, 30, 40 or 50 wt %, or the same values of another specified unit.

The term “crude oil” as used herein refers to petroleum extracted from geologic formations in its unrefined form. Crude oil that is suitable as the source material for the processes herein include Arabian Heavy, Arabian Light, Arabian Extra Light, other Gulf crudes, Brent, North Sea crudes, North and West African crudes, Indonesian, Chinese crudes, North or South American crudes, Russian and Central Asian crudes, or mixtures thereof. The crude petroleum mixtures can be whole range crude oil or topped crude oil. As used herein, “crude oil” also refers to such mixtures that have undergone some pre-treatment such as water-oil separation; and/or gas-oil separation; and/or desalting; and/or stabilization. In certain embodiments, crude oil refers to any of such mixtures having an API gravity (ASTM D287 standard), of greater than or equal to about 20°, 30°, 32°, 34°, 36°, 38°, 40°, 42° or 44°.

The term “condensates” refers to hydrocarbons separated from natural gas stream. As used herein, “condensates” also refers to such mixtures that have undergone some pre-treatment such as water-oil separation; and/or gas-oil separation; and/or desalting; and/or stabilization. In certain embodiments, condensates refer to any of such mixtures having an API gravity (ASTM D287 standard), of greater than or equal to about 45, 50, 60, or 65°.

The acronym “LPG” as used herein refers to the well-known acronym for the term “liquefied petroleum gas,” and generally is a mixture of C3-C4 hydrocarbons. In certain embodiments, these are also referred to as “light ends.”

As used herein, all boiling point ranges relative to hydrocarbon fractions derived from crude oil via atmospheric and/or vacuum distillation shall refer to True Boiling Point values obtained from a crude oil assay, or a commercially acceptable equivalent.

The term “naphtha” as used herein refers to hydrocarbons having a nominal boiling range of about 20-205, 20-193, 20-190, 20-180, 20-170, 32-205, 32-193, 32-190, 32-180, 32-170, 36-205, 36-193, 36-190, 36-180 or 36470° C.

The term “light naphtha” as used herein refers to hydrocarbons having a nominal boiling range of about 20-110, 20-100, 20-90, 20-88, 32-110, 32-100, 32-90, 32-88, 36-110, 36-100, 36-90 or 36-88° C.

The term “heavy naphtha” as used herein refers to hydrocarbons having a nominal boiling range of about 90-205, 90-193, 90-190, 90-180, 90-170, 93-205, 93-193, 93-190, 93-180, 93-170, 100-205, 100-193, 100-190, 100-180, 100-170, 110-205, 110-193, 110-190, 110-180 or 110-170° C.

In certain embodiments, naphtha, light naphtha and/or heavy naphtha refer to such petroleum fractions obtained by crude oil distillation, or distillation of intermediate refinery processes as described herein.

The modifying term “straight run” is used herein having its well-known meaning, that is, describing fractions derived directly from the atmospheric distillation unit, optionally subjected to steam stripping, without other refinery treatment such as hydroprocessing, fluid catalytic cracking or steam cracking. An example of this is “straight run naphtha” and its acronym “SRN” which accordingly refers to “naphtha” defined above that is derived directly from the atmospheric distillation unit, optionally subjected to steam stripping, as is well known.

In certain embodiments, the term “middle distillate” is used with reference to one or more straight run fractions from the atmospheric distillation unit, for instance containing hydrocarbons having a nominal boiling range of about 160-400, 160-380, 160-370, 160-360, 160-340, 170-400, 170-380, 170-370, 170-360, 170-340, 180-400, 180-380, 180-370, 180-360, 180-340, 190-400, 190-380, 190-370, 190-360, 190-340, 193-400, 193-380, 193-370, 193-360, or 193-340° C. In embodiments in which other terminology is used herein, the middle distillate fraction can also include all or a portion of AGO range hydrocarbons, all or a portion of kerosene, all or a portion of medium AGO range hydrocarbons, and/or all or a portion of heavy kerosene range hydrocarbons. In additional embodiments, the tell “middle distillate” is used to refer to fractions from one or more integrated operations boiling in this range.

The tem “atmospheric residue” and its acronym “AR” as used herein refer to the bottom hydrocarbons having an initial boiling point corresponding to the end point of the AGO or middle distillate range hydrocarbons, and having an end point based on the characteristics of the crude oil feed.

The term “vacuum gas oil” and its acronym “VGO” as used herein refer to hydrocarbons having a nominal boiling range of about 370-565, 370-550, 370-540, 370-530, 370-510, 400-565, 400-550, 400-540, 400-530, 400-510, 420-565, 420-550, 420-540, 420-530 or 420-510° C.

The term “vacuum residue” and its acronym “VR” as used herein refer to the bottom hydrocarbons having an initial boiling point corresponding to the end point of the VGO range hydrocarbons, and having an end point based on the characteristics of the crude oil feed.

The term “fuels” refers to crude oil-derived products used as energy carriers. Fuels commonly produced by oil refineries include, but are not limited to, gasoline, jet fuel, diesel fuel, fuel oil and petroleum coke. Unlike petrochemicals, which are a collection of well-defined compounds, fuels typically are complex mixtures of different hydrocarbon compounds.

The terms “aromatic hydrocarbons” or “aromatics” is very well known in the art. Accordingly, the term “aromatic hydrocarbon” relates to cyclically conjugated hydrocarbons with a stability (due to delocalization) that is significantly greater than that of a hypothetical localized structure (for example, Kekule structure). “Aromatic hydrocarbons” or “aromatics” can refer to cyclically conjugated hydrocarbons having a single ring or multiple rings. A common method for determining aromaticity of a given hydrocarbon is the observation of diatropicity in its 1H NMR spectrum, for example the presence of chemical shifts in the range of from 7.2 to 7.3 ppm for benzene ring protons.

As used herein, the term “aromatic products” includes one or more of: C6-C8 aromatics including benzene, toluene and mixed xylenes (commonly referred to as BTX); C6-C8 aromatics including benzene, toluene, ethylbenzene and mixed xylenes (commonly referred to as BTEX), C6 and C8 aromatics including benzene and paraxylene; and any combination thereof. These aromatic products have a premium chemical value.

The term “unconverted oil” and its acronym “UCO,” is used herein having its known meaning, and refers to a highly paraffinic and naphthenic fraction from a hydrocracker with a low nitrogen, sulfur and nickel content and including hydrocarbons having a nominal boiling range with an initial boiling point corresponding to the end point of the AGO range hydrocarbons, in certain embodiments the initial boiling point in the range of about 340-370° C., for instance about 340, 360 or 370° C., and an end point in the range of about 51.0-565° C., for instance about 540, 550 or 565° C. UCO is also known in the industry by other synonyms including “hydrowax.”

The term “C #hydrocarbons” or “C #”, is used herein having its well-known meaning, that is, wherein “#” is an integer value, and means hydrocarbons having that value of carbon atoms. The term “C #+ hydrocarbons” or “C·+” refers to hydrocarbons having that value or more carbon atoms. The term “C #− hydrocarbons” or “C #−” refers to hydrocarbons having that value or less carbon atoms. Similarly, ranges are also set forth, for instance, C1-C3 means a mixture comprising C1, C2 and C3.

The term “petrochemicals” or “petrochemical products” refers to chemical products derived from crude oil that are not used as fuels. Petrochemical products include olefins and aromatics that are used as a basic feedstock for producing chemicals and polymers. Typical olefinic petrochemical products include, but are not limited to, ethylene, propylene, butadiene, butylene-1, isobutylene, isoprene, cyclopentadiene and styrene. Typical aromatic petrochemical products include, but are not limited to, benzene, toluene, xylene, and ethyl benzene.

The term “olefin” is used herein having its well-known meaning, that is, unsaturated hydrocarbons containing at least one carbon-carbon double bond. In plural, the term “olefins” means a mixture comprising two or more unsaturated hydrocarbons containing at least one carbon-carbon double bond. In certain embodiments, the term “olefins” relates to a mixture comprising two or more of ethylene, propylene, butadiene, butylene-1, isobutylene, isoprene and cyclopentadiene. The term “light olefins” or “light olefin products” is used herein having its well-known meaning, that is: ethylene and propylene; or ethylene, propylene, butylene and butadiene.

The term “make-up hydrogen” is used herein with reference to hydroprocessing zones to refer to hydrogen requirements of the zone that exceed recycle from conventionally integrated separation vessels, in certain embodiments as used herein all or a portion of the make-up hydrogen in any given hydroprocessing zone or reactor within a zone is from gases derived from the petrochemical production operation(s) in the integrated processes and systems.

The term “light cycle oil” and its acronym “LCO” as used herein refers to the light cycle oil produced by FCC units. The nominal boiling range for this stream is, for example, in the range of about 215-350, 216-350, 220-350, 215-343, 216-343, 220-343, 215-330, 216-330 or 220-330° C. LCO, directly from FCC separation or after hydrotreating, is conventionally used in diesel blends depending on the diesel specifications, or as a cutter to the fuel oil tanks for a reduction in the viscosity and sulfur contents.

The term “heavy cycle oil” and its acronym “HCO” as used herein refer to the heavy cycle oil which is produced by fluid catalytic cracking units. The nominal boiling range for this stream is, for example, in the range of about 330-530, 330-510, 343-530, 343-510, 350-530 or 350-510° C. HCO is conventionally used in an oil flushing system within the process. Additionally, HCO is conventionally used to partially vaporize debutanizer bottoms and for recycle as a circulating reflux to the main fractionator in the fluid catalytic cracking unit.

The term “cycle oil” is used herein to refer to a mixture of LCO and HCO.

The term “slurry oil” is used herein to refer to the heaviest fraction which remain as a bottoms fraction after fluid catalytic cracking units. Slurry oil typically contains solid catalyst particles and/or fines. The hydrocarbon mixture of slurry oil is highly aromatic, high boiling, dense liquid. Slurry oil is conventionally used as fuel oil or can be processed (after removal of solid catalyst particles and/or fines).

It is to be understood that like numerals in the drawings represent like elements through the several figures, and that not all components and/or steps described and illustrated with reference to the figures are required for all embodiments or arrangements. Further, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “including,” “comprising,” or “having,” “containing,” “involving,” and variations thereof herein, when used in this specification, specify the presence of stated features, integers, steps, operations, elements, and/or components, but do not preclude the presence or addition of one or more other features, integers, steps, operations, elements, components, and/or groups thereof. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Notably, the figures and examples above are not meant to limit the scope of the present disclosure to a single implementation, as other implementations are possible by way of interchange of some or all the described or illustrated elements. Moreover, where certain elements of the present disclosure can be partially or fully implemented using known components, only those portions of such known components that are necessary for an understanding of the present disclosure are described, and detailed descriptions of other portions of such known components are omitted so as not to obscure the disclosure. In the present specification, an implementation showing a singular component should not necessarily be limited to other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). It is to be understood that dimensions discussed or shown are drawings accordingly to one example and other dimensions can be used without departing from the disclosure.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

1. A process to assess efficacy of at least one property of regenerated fluid catalytic cracking (FCC) catalyst, comprising:
withdrawing a minor portion of the regenerated FCC catalyst from a main FCC zone for testing;
introducing the withdrawn regenerated FCC catalyst to a testing zone including at least one test reactor;
introducing at least one test feedstock to the at least one test reactor containing the regenerated FCC catalyst for reaction to produce effluent test hydrocarbons and used test catalyst; and
assessing at least one property of effluent test hydrocarbons, used test catalyst, or both effluent test hydrocarbons and used test catalyst.

2. The process as in claim 1, wherein the minor portion comprises 0.0001-2 wt % of regenerated FCC catalyst from the main FCC zone.

3. The process as in claim 1, wherein:
the main FCC zone includes at least a regeneration zone and a main FCC reactor;
regenerated FCC catalyst is passed from the regeneration zone to the main FCC reactor; and
the minor portion of regenerated FCC catalyst that is withdrawn for testing is from the regeneration zone.

4. The process as in claim 3, wherein the regenerated FCC catalyst is withdrawn at predefined intervals, continuously, continuously with intermittent interruption, continuously with interruption or at predefined intervals.

5. The process as in claim 3, wherein the testing zone comprises a scaled down test reactor that is 1-25, 1-20, 1-15, or 1-10% of the size of the main FCC reactor.

6. The process as in claim 1, wherein:
the at least one test feedstock comprises a portion of a hydrocarbon feedstock used in the main FCC zone;
the test reactor operates with parameters that differ from corresponding operational parameters of the main FCC zone; and
the parameters include catalyst to oil ratio, temperature, residence time, injection location of the one or more of the at least one hydrocarbon feedstocks and/or withdrawn regenerated FCC catalyst, or combinations of the parameters.

7. The process as in claim 1, wherein the at least one test feedstock comprises a hydrocarbon feedstock that is different than that used in the main FCC zone obtained from distillation, delayed coking, hydrocracking or hydrotreating units.

8. The process as in claim 1, wherein an additional catalyst or catalyst additive is introduced to the test reactor.

9. The process as in claim 1, wherein:
the testing zone comprises a plurality of test reactors in parallel; and
each test reactor receives regenerated FCC catalyst and one or more of the at least one test feedstocks.

10. The process as in claim 9, wherein:
the plurality of test reactors includes a first reactor and a second reactor;
during reaction the first reactor operates at a first set of parameters the second reactor operates at a second set of parameters; and
the first set of parameters and the second set of parameters contain at least one parameter that is different.

11. The process as in claim 10, wherein the at least one parameter of the first set of parameters and the second set of parameters which are different include one or more of catalyst to oil ratio, temperature, residence time and injection location of the one or more of the at least one hydrocarbon feedstocks.

12. The process as in claim 9, wherein:
the at least one test feedstock comprises at least two test feedstocks including a first test feedstock and a second test feedstock which are different from one another;
the plurality of test reactors includes a first reactor and a second reactor; and
the first test feedstock is introduced into the first reactor and the second test feedstock is introduced into the second reactor.

13. The process as in claim 12, wherein the first test feedstock comprises a hydrocarbon mixture corresponding to that used in the main FCC zone from which the regenerated FCC catalyst for testing is obtained, and the second test feedstock comprises a hydrocarbon mixture that differs from that used in the main FCC zone from which the regenerated FCC catalyst for testing is obtained.

14. The process as in as in claim 1, wherein:
the testing zone comprises a plurality of test reactors in parallel, wherein at least one test reactor receives regenerated FCC catalyst and one or more of the at least one test feedstocks; and
at least one test reactor receives fresh FCC catalyst and optionally fresh FCC catalyst additives.

15. The process as in claim 1, wherein:
the regenerated FCC catalyst for testing is heated; and
the heated regenerated FCC catalyst for testing heats the at least one test feedstock in the at least one test reactor.

16. The process as in claim 1, wherein the test reactor operates at a temperature in the range of about 300-700° C.

17. The process as in claim 1, wherein the test reactor operates with a ratio of the regenerated FCC catalyst for testing to the at least one hydrocarbon feedstock of 1:1 to 60:1 on a weight basis.

18. The process as in claim 1, wherein the testing zone further comprises a solid-gas-liquid separation system.

19. The process as in claim 1, wherein:
at least one property of effluent test hydrocarbons is assessed; and
the at least one property of effluent test hydrocarbons is selected from the group consisting of heteroatom content, simulated distillation data, hydrogen content, density, PONA content, PIONA, relative yield fractions, light olefin content, hydrogen sulfide content, Reid vapor pressure (RVP), octane number, cetane index, smoke point and pour point.

20. The process as in claim 1, wherein:
at least one property of used test catalyst is assessed; and
the at least one property of used test catalyst is selected from the group consisting of coke content, heteroatom content and mechanical integrity.
